(19) United States
(12) Patent Application Publication
(10) Pub. No.: US 2024/0342413 A1
(43) Pub. Date: Oct. 17, 2024

(54) DUAL PURPOSE RESPIRATORY TREATMENT DEVICE

(71) Applicant: Jeffrey Carl Cowgill, Vancouver, WA (US)

(72) Inventor: Jeffrey Carl Cowgill, Vancouver, WA (US)

(21) Appl. No.: 18/301,933

(22) Filed: Apr. 17, 2023

(51) Int. Cl. A61M 16/04

(52) U.S. Cl. CPC A61M 16/0463

(57) ABSTRACT

A portable, patient operated respiratory device that incorporates both an airway clearance device and a phlegm elimination system in a single unit.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to the medical field, and more particularly to respiratory technology.

### BACKGROUND

Respiratory treatments vary considerably as do the different manufacturer's devices used to facilitate the treatments. Generally, these treatments accomplish two goals. First, it helps strengthen the patient's lungs and second, it clears the phlegm and mucus from the to the patient's airways. This is accomplished with the patient undergoing separate regimens of positive exhalation pressure (PEP) treatments, possibly with a subsequent medicament inhalation (drug delivery). The PEP treatment requires the patient to exhale through any of a plethora of PEP devices that send a resultant pulsation pressure wave back down into their lungs. If this pressure wave has sufficient power, it will dislodge the phlegm from their airways. When enough cycles have been performed, the patient's breathing ability is improved as their lungs are cleared of phlegm to the point where they can undergo an inhalation treatment and receive a medicated aerosol (generally inhaled antibiotics, bronchodilators, corticosteroids) to further increase their lung capacity and ease their labored breathing.

There are several problems encountered by patients using such PEP devices. When the phlegm is brought out of the patient's airways it must exit the mouth and needs a place to be expelled. There is not always a handy place nearby to receive the phlegm. Patients are often elderly, weak or partially incapacitated. Because of their diminished lung capacity, successful exhalations (events) often leaves them out of breath and gasping for air. At this time, dealing with a mouth full of phlegm, while holding a PEP device and trying to catch one's breath is daunting for many. This also stops the rhythm of the PEP routine which is important as the effect of each exhalation builds on itself. A repeated pattern of inhalation and exhalation is necessary to gain the full medical effect of the PEP device. This should not be interrupted.

If the PEP device is contaminated with mucus it may cease to function or at the least, require intensive cleaning subject to prevent bacterial growth. There are vacuum units to deal with the phlegm but switching between devices increases the treatment complexity and robs precious time between breaths.

For these reasons, self-treatment with a PEP device is not a possibility, or if attempted will not provide the full beneficial effects.

Thus, a novel device that handles the full spectrum of respiratory training and is able to be administered by the patient alone would fulfill a long felt need in the medical industry. This new invention utilizes and combines known and new technologies in a unique and novel configuration to overcome the aforementioned problems and accomplish this.

### BRIEF SUMMARY

A suction adaptor integrated in, and/or on, a PEP device for patients with severe airway clearance issues that protects against potential airway aspiration and can be used for in hospital use and home care.

In accordance with various embodiments, a PEP device is provided that utilizes a separate pathway external to the device's respiratory pathway to eliminate the phlegm developed.

In one aspect, a self-administered respiratory treatment device that allows a patient to maintain the regular, rhythmed breathing to maximize the benefit of the PEP technology.

In another aspect, a self-administered respiratory treatment device incorporating a PEP device and an integrated suction unit that can be quickly employed by the patient is provided.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a side perspective view of the first embodiment respiratory treatment device;

FIG. 2 is a front view of the first embodiment respiratory treatment device;

FIG. 3 is a side view of a vacuum unit;

FIG. 4 is a side view of a first embodiment respiratory treatment device in use;

FIG. 5 is a side perspective view of a second embodiment respiratory treatment device;

FIG. 6 is a side view of a second embodiment respiratory treatment device in use; and

FIG. 7 is a front view of a second embodiment respiratory treatment device.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The accompanying drawings are not necessarily drawn to scale. The examples provided are for illustrative purposes and are not intended to limit the scope of the invention.

It will be understood that when an element or layer is referred to as being “on,” “coupled to,” or “connected to” another element or layer, it can be directly on, directly coupled to or directly connected to the other element or layer, or intervening elements or layers may be present. In contrast, when an element is referred to as being “directly on,” “directly coupled to,” or “directly connected to” another element or layer, there are no intervening elements or layers present. Like numbers refer to like elements throughout. As used herein, the term “and/or” includes any and all combinations of one or more of the associated listed items.

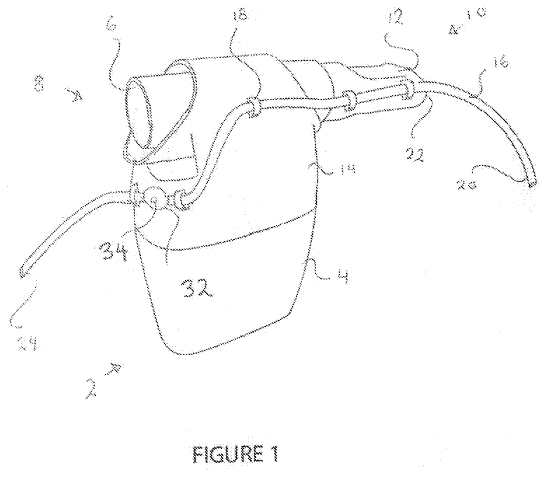

The present invention relates to a novel design for a respiratory treatment device that blends together complimentary devices used in the treatment of respiratory diseases. Looking at FIG. 1, the first embodiment dual purpose respiratory device 2 can best be seen. It consists of a sterilizeable polymer body 4 with an internal cavity that houses an airway clearance device that develops a pulsation back pressure wave in a patients exhaled stream of air, that is transmitted back down to the patient's lungs. In the preferred embodiment this airway clearance device will use any of a series of known Positive Expiration Pressure (PEP) devices that may use flattened air tubes, moveable air restrictors, flapper valves, or the functional mechanical equivalents to generate the back pressure wave. These air pulses dislodge the phlegm from the patients lungs, bronchi, and trachea so that it may be coughed up and out into the mouth. (These different PEP device's mechanical components are well known in the industry and are not illustrated herein but are indicated as contained within the polymer body 4.) Once removed, the airway pathway is clearer allowing for easier respiration.

It is to be understood that depending on which style of airway clearance device is used, the physical configuration of the polymer body will differ, however, all these devices share commonalities that allow them to be modified consistent with the design provided herein to accomplish the same end result.

The body 4 of the dual purpose respirator treatment device 2 has a front end 8 with an exhaled air exit 6, and a back end 10 with a patient mouthpiece 12 having a exhaled air entrance 11. The exhaled air entrance 11 directs patient exhaled air into a front end of the airway clearance device and a back end of the airway clearance device directs air to the exhaled air exit 6.

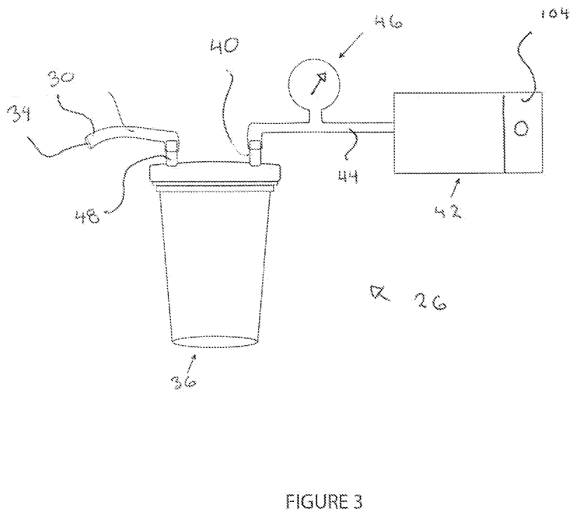

Removably attached to at least one exterior side face 14 of the body 4 and along the mouthpiece 12, is a flexible suction catheter 16. This catheter 16 is held in position and has its proximal end extendable beyond the mouthpiece by a catheter securement means which may be openable tubing clips 18 or a section of tubing. In the preferred embodiment, this catheter 16 runs through a series of openable tubing clips 18 that are affixed to the side 14 and mouthpiece, and are sized for the frictional retention of the catheter 16. Its profile is tight to the body's side 14 and mouthpiece 12, to minimize snagging of the catheter. The proximal end 20 of the catheter is open and extends beyond the end 22 of the mouthpiece 20 approximately one to three inches, so as to reside in the patient's mouth when the patient is exhaling into the device 2. The distal end 24 of the catheter 16 is connected to a suction tube 30 of a vacuum unit 26. (See FIG. 3) There is a tube connector 28 used to connect the catheter 16 to the suction tube 30. Using a short catheter 16 that couples to the suction tube 30 allows for a smaller diameter, shorter catheter which is easier to clean and replace. In alternate embodiments there may be but a single suction tube that can be attached to the device 2 with the clips 18, acting as a long catheter, although this is not the preferred design.

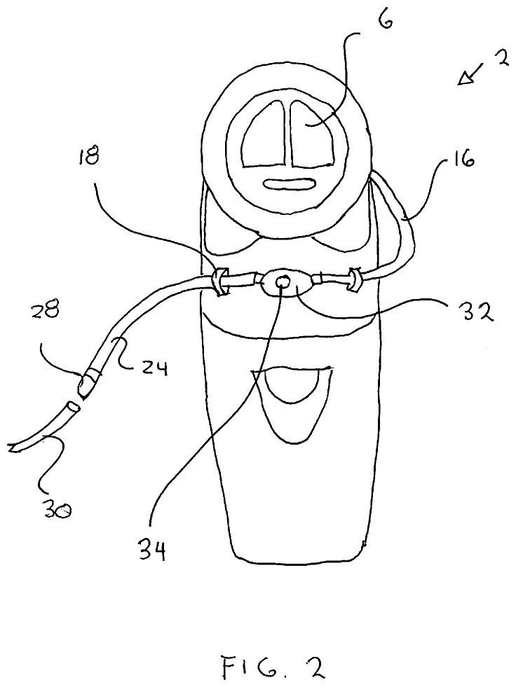

Looking at FIGS. 1 and 2 it can be seen that between the catheter's proximal end 20 and distal end 24, there is an variable suction control means 32. This suction control means 32, is adjacent an orifice 100 formed through the catheter 16 and when depressed, occludes an orifice 100 (shown dotted in phantom) formed in the side wall 106 of the catheter which causes the vacuum developed by the vacuum unit 26 to draw in air from the catheter's proximal end 20 so as to suction out the phlegm the patient coughs up into their mouth. Essentially, this suction control means 32 is a moveable valve that can seal or allow variable amount of air to be drawn in through an orifice 100 in the catheter 16 or through the proximal end of the catheter 16. In the preferred embodiment, this suction control means 32 is a paddle arm that the patient may squeeze with the fingers of the device holding hand, to operate. In the preferred embodiment this suction control means 32 has an adjustable volume control 34 that can be tactilely manipulated by the patient or caregiver to limit the amount of suction or the volume that can be drawn per unit time from the proximal end of the catheter 16. (The orifice 100 may be partially sealed.) The level of suction can be regulated to a degree by the level of vacuum developed and waiting in the vacuum chamber 36. This is indicated at the vacuum gauge 46 which resides between the vacuum pump and the vacuum chamber 36 and is regulated by adjustment of the vacuum pump controller. This is important as the patient will close their mouth on the proximal end 20 of the catheter 16 to suction away the phlegm, but once removed, the suction will act to collapse the patient's cheeks which is a very uncomfortable feeling. The suction control means 32 is located and secured on the body 4 at a location on the device 2 that coincides with where the patient's fingertips reside when they grasp the device 2 for use. In the preferred embodiment this is at the front side. A paddle style

Looking at FIG. 3 the vacuum unit 26 can best be explained. The vacuum unit 26 in its simplest configuration, has a vacuum chamber 36, sealed at the top by a lid 38 with two tubing connectors, a pump tubing connector 40 and a suction tubing connector 48. The pump tubing connector 40 is connected to a section of tubing 44 that is connected directly to the suction side of the vacuum pump 42. There is a vacuum gauge 46 tapped off of this section of tubing 44 indicating the level of vacuum developed by the vacuum unit 26. There is a vacuum pump controller 104 incorporated into the vacuum pump 42 that sets the level of vacuum developed. The suction tubing connector 48 is connected to the suction tubing 30 which is connected to the catheter 16. The vacuum pump 42 evacuates the air in the vacuum chamber 36 and in the suction tubing up to the orifice 100 located in the catheter 16 at the suction control means 32. Once this orifice is occluded by depressing the paddle of the suction control means 32, the vacuum is drawn from the proximal end 20 of the catheter 16.

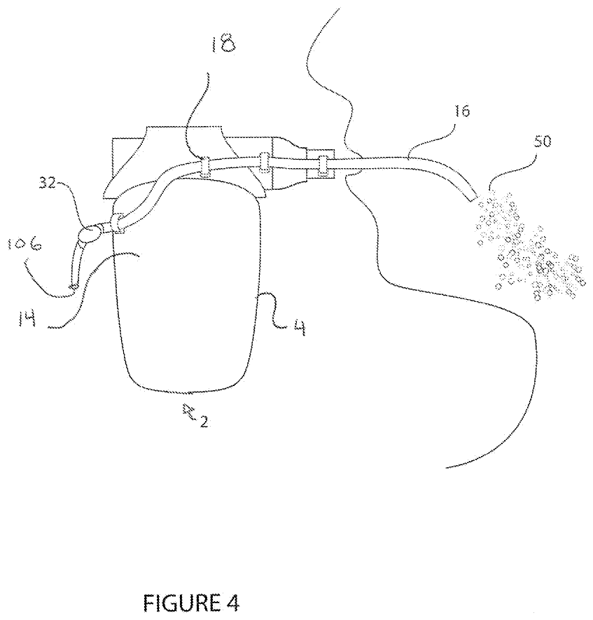

In operation, when the suction control means 32 is operated, phlegm 50 can be drawn from the proximal end 20 of the catheter 16 and deposited into the vacuum chamber 36. FIG. 4 illustrates this procedure up to the connector 28.

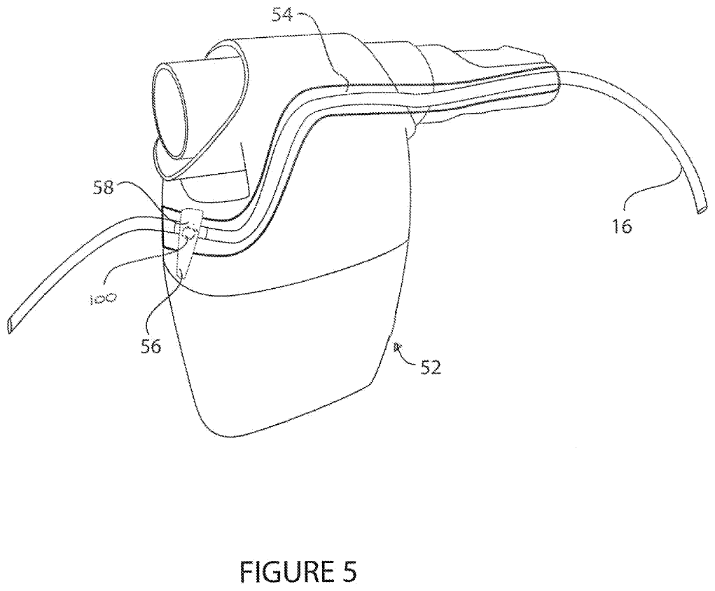

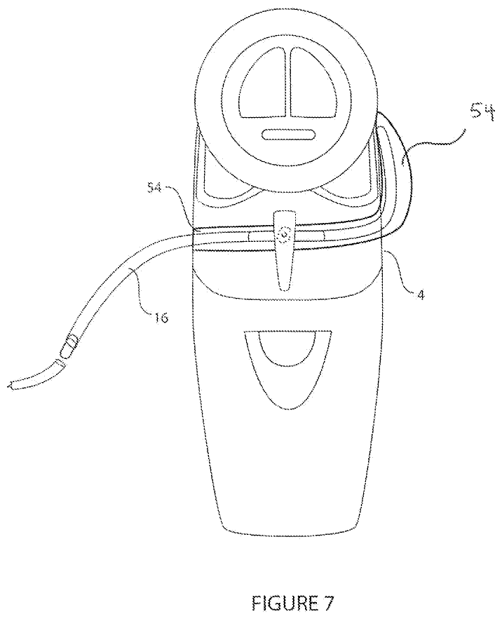

FIGS. 5-7 illustrate the second embodiment dual purpose respiratory device 52. This varies from the first embodiment dual purpose respiratory device 2 in that the catheter securement means is an enclosed translucent channel 54 formed from a section of clear tubing on the body that frictionally accepts and retains the catheter 16. Since the tubing is also clear, any blockage in the catheter 16 as well as the general cleanliness of the catheter 16, can be seen and the catheter 16 replaced as necessary.

In the first and second embodiments, the suction control means 32 is swivel arm mounted to the body with a pivot pin 58 that allows the suction control means 32 to be pivoted out of the way for the replacement of a new catheter 16, and pivoted over the catheter 16 and its orifice 100 for operation and to further ensure the retention of the catheter 16. Once the catheter's proximal end 30 is extended to the proper distance from the end of the patient mouthpiece 12, the catheter 16 is locked in place with this swivel arm 56. In the second embodiment, there is a partial section of the translucent channel 54 removed under the suction control means 32 such that the suction control means 32 (swivel arm) may be depressed like a paddle arm to variably occlude the orifice in the catheter to regulate the amount of suction at the proximal end of the catheter.

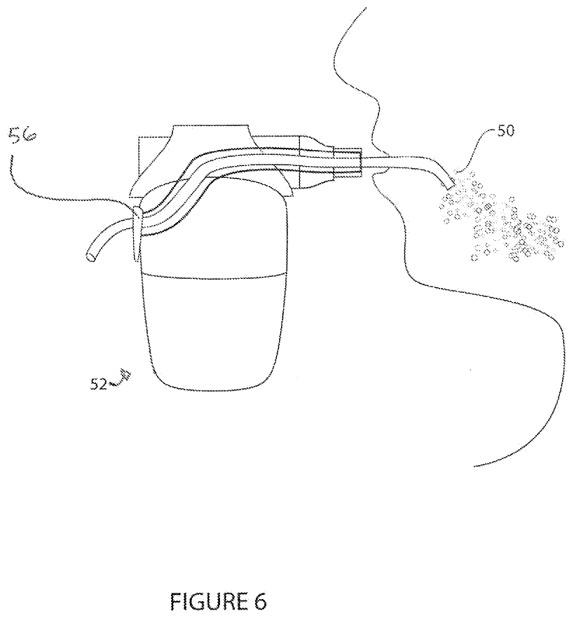

As shown by FIG. 6, the operation of this second embodiment remains the same as that of the first embodiment. Once PEP therapy is initiated, the patient may elicit a cough and in turn bring up secretions. Then the patient or caregiver can move the proximal end of the catheter 16 as far back as the oropharynx as necessary and open the suction control means 32 to to quickly remove secretions safely.

In both embodiments, the devices 2 and 52 are designed for patients with severe airway clearance issues. It protects against potential airway aspiration and does not interfere with PEP treatments. Most importantly, it allows patients to complete their own respiratory treatments alone. Prior to this there was always the potential for choking when large volumes of phlegm were brought up.

While certain features and aspects have been described with respect to exemplary embodiments, one skilled in the art will recognize that numerous modifications are possible. As such, this detailed description and accompanying material is intended to be illustrative only, and should not be taken as limiting the scope of the inventive concept. What is claimed as the invention, therefore, is all such modifications as may come within the scope and spirit of the following claims and equivalents thereto.

Having thus described the invention, what is claimed as new and desired to be secured by Letters Patent is as follows:

1. A patient operated airway clearance and phlegm elimination device, comprising:
a sterilizeable polymer body with an internal cavity that houses an airway clearance device, said body having a front end with an exhaled air exit, and a back end with a patient mouthpiece having a exhaled air entrance;
a catheter removably attached to said polymer body, said catheter having a proximal end and a distal end,
an orifice formed through a side wall of said catheter between said proximal end and said distal end
a variable suction control means positioned adjacent said orifice, said suction control means moveable between a position that occludes or does not occlude said orifice; and
a vacuum system connected to said distal end of said catheter.

2. The patient operated airway clearance and phlegm elimination device of claim 1 wherein said proximal end of said catheter adjustably extends beyond said patient mouthpiece.

3. The patient operated airway clearance and phlegm elimination device of claim 2 further comprising:
a catheter securement means selected from the set of group consisting of openable tubing clips or a section of tubing;
wherein said catheter securement means is affixed to an exterior face of said body and restrains said catheter thereon said body.

4. The patient operated airway clearance and phlegm elimination device of claim 3 wherein said sections of tubing are clear.

5. The patient operated airway clearance and phlegm elimination device of claim 4 wherein said suction control means is a swivel arm mounted to said body with a pivot pin.
